(19) United States
(12) Patent Application Publication
(10) Pub. No.: US 2005/0014700 A1
(43) Pub. Date: Jan. 20, 2005

(54) Cbi analogues of cc-1065 and the duocarmycins

(76) Inventor: Dale L. Boger, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(21) Appl. No.: 10/489,006

(22) PCT Filed: Sep. 9, 2002
(86) PCT No.: PCT/US02/28749

(30) Foreign Application Priority Data
Sep. 7, 2001 (US) 60318179

(51) Int. Cl. A61K 38/04; C07K 5/04; C07D 417/02; C07D 413/02; C 07D 4 3/02

(52) U.S. Cl. 514/19; 548/181; 548/304.7; 548/420; 548/217; 548/427

(57) ABSTRACT

132 CBI analogues of CC-1 065 and the duocarmycins having dimeric monocyclic, bicyclic, and tricyclic heteroaromatics substituents were synthesized by a parallel route. The resultant analogues were evaluated with respect to their catalytic and cytotoxic activities. The relative contribution of the various dimeric monocyclic, bicyclic, and tricyclic heteroaromatics substituents within the DNA binding domain were characterized. Several of the resultant CBI analogues of CC-1065 and the duocarmycins were characterized as having enhanced catalytic and cytotoxic activities and were identified as having utility as anti-cancer agents.

### DESCRIPTION

1. Field of Invention

The present application relates to CBI analogues of CC-1065 and the duocarmycins and to their synthesis and use as cytotoxic agents. More particularly, the present invention relates to CBI analogues of CC-1065 and the duocarmycins having dimeric monocyclic, bicyclic, and tricyclic heteroaromatics substituents and to their synthesis and use as cytotoxic agents.

2. Background

CC-1065 (1) and the duocarmycins (2 and 3) are among the most potent antitumor antibiotics discovered to date (Hanka, L. J., et al., Antibiot. 1978, 31, 1211; and Boger, D. L. Chemtracts: Org. Chem. 1991, 4, 329). These compounds have been shown to derive their biological activity through the sequence selective alkylation of duplex DNA (FIG. 1) (Warpehoski, M. A. In Advances in DNA Sequence Specific Agents; Hurley, L. H., Ed.; JAI Press: Greenwich, CT, 1992; Vol. 1, p 217; Hurley, L. H., et al., Chem. Res. Toxicol. 1988, 1, 315; Boger, D. L., et al., Angew. Chem., Int. Ed. Engl. 1996, 35, 1438; and Boger, D. L., et al., Proc. Natl. Acad. Sci. U.S.A. 1995, 92, 3642). An extensive series of studies have defined the nature of the alkylation reaction, which proceeds by adenine N3 addition to the least substituted cyclopropane carbon of the left-hand alkylation subunit, and the alkylation sequence selectivity (Hurley, L. H., et al., Science 1984, 226, 843; Hurley, L. H., et al., Biochemistry 1988, 27, 3886; Hurley, L. H., et al., J. Am. Chem. Soc. 1990, 112, 4633; Boger, D. L., et al., Bioorg. Med. Chem. 1994, 2, 115; Boger, D. L., et al., J. Am. Chem. Soc. 1990, 112, 4623; Boger, D. L., et al., J. Org. Chem. 1990, 55, 4499; Boger, D. L., et al., J. Am. Chem. Soc. 1990, 112, 8961; Boger, D. L., et al., J. Am. Chem. Soc. 1991, 113, 6645; Boger, D. L., et al., Am. Chem. Soc. 1993, 115, 9872; Boger, D. L., et al., J. Am. Chem. Soc. 1994, 116, 1635; and Asai, A., et al., J. Am. Chem. Soc. 1994, 116, 4171). For the natural enantiomers, this entails 3′ adenine N3 alkylation with binding across a 3.5-4(duocarmycins) or 5 (CC-1065) base-pair AT-rich site (e.g. 5′-AAAAA), whereas the unnatural enantiomers bind in the reverse 5′→3′ direction (e.g. 5′-AAAAA) across analogous 3.5-5 base-pair AT-rich sites (Boger, D. L., et al., Angew. Chem., Int. Ed. Engl. 1996, 35, 1438; and Boger, D. L., et al., Proc. Natl. Acad. Sci. U.S.A. 1995, 92, 3642). An alternative way of visualizing this behavior of the two enantiomers is that from a common bound orientation and within a common binding site, they alkylate adenine on complementary strands of duplex DNA at sites offset by one base-pair

$$( e . g . , \begin{matrix}{5}^{′} \text{-} AATTA ( natural ) \\ {3}^{′} \text{-} TTAAT ( unnatural )\end{matrix} )$$

(Smith, J. A., et al., J. Mol. Biol. 2000, 300, 1195; Eis, P.S., et al., J. Mol. Biol. 1997, 272, 237; and Schnell, J. R., et al., J. Am. Chem. Soc. 1999, 121, 5645). Early studies demonstrated that the right-hand segment(s) of the natural products effectively deliver the alkylation subunit to AT-rich sequences of duplex DNA increasing the selectivity and efficiency of DNA alkylation (Boger, D. L., et al., Chem.-Biol. Interact. 1990, 73, 29). Because this preferential AT-rich noncovalent binding affinity and selectivity, like that of distamycin and netropsin (Johnson, D. S., et al., In Supramolecular Chemistry; and Lehn, J.-M., Ed.; Pergamon Press: Oxford, 1996; Vol. 4, p 73), is related to the deeper and narrower shape of the AT-rich minor groove, it is often referred to a shape-selective recognition. However, it is only in more recent studies that it has become apparent that the DNA binding domain also plays an important role in catalysis of the DNA alkylation reaction (Boger, D. L., et al., Bioorg. Med. Chem. 1997, 5, 263; and Boger, D. L., et al., Acc. Chem. Res. 1999, 32, 1043). Because this is also related to the shape characteristics of the minor groove and results in preferential activation in the narrower, deeper AT-rich minor groove, this is referred to as shape-dependent catalysis (Boger, D. L., et al., Bioorg. Med. Chem. 1997, 5, 263; and Boger, D. L., et al., Acc. Chem. Res. 1999, 32, 1043). This catalysis may be derived from a DNA binding-induced conformational change in the agents which adopt a helical DNA bound conformation requiring a twist in the amide linking of the alkylation subunit and the first DNA binding subunit. This conformational change serves to partially deconjugate the stabilizing vinylogous amide, activating the cyclopropane for nucleophilic attack. For activation, this requires a rigid, extended (hetero)aromatic N2-amide substituent (Boger, D. L., et al., J. Am. Chem. Soc. 1997, 119, 4977; Boger, D. L., et al., J. Am. Chem. Soc. 1997, 119, 4987; and Boger, D. L., et al., Bioorg. Med. Chem. 1997, 5, 233) and the shape, length, and strategically positioned substituents on the first DNA binding subunit can have a pronounced effect on the DNA alkylation rate and efficiency and the resulting biological properties of the agents.

The combination of the effects is substantial. The DNA alkylation rate and efficiency increases approximately 10,000-fold and the resulting biological potency also increases proportionally 10,000-fold when comparing simple N-acetyl or N-Boc derivatives of the alkylation subunits, which lack the DNA binding domain, with 1-3. In three independent studies, the DNA binding subunit contribution to DNA alkylation rate could be partitioned into that derived from an increased binding selectivity/affinity and that derived from a contribution to catalysis of the DNA alkylation reaction. The former was found to increase the rate approximately 10-100-fold, whereas the latter increases the rate approximately 1000-fold indicating a primary importance (Boger, D. L., et al., J. Am. Chem. Soc. 2000, 122, 6325; Boger, D. L., et al., J. Org. Chem. 2000, 65, 4088; and Boger, D. L., et al., J. Am. Chem. Soc., in press).

Throughout these investigations, the complementary roles of the DNA binding subunits have been examined with relatively limited numbers of compounds and no systematic study has been disclosed. Moreover, there is some confusion in the disclosures as to the relative effectiveness of the distamycin/lexitropsin substitutions for the DNA binding subunits, both with regard to DNA alkylation selectivity and alkylation efficiency (Wang, Y., et al., Heterocycles 1993, 36, 1399; Fregeau, N. L., et al., J. Am. Chem. Soc. 1995, 117, 8917; Wang, Y., et al., Anti-Cancer Drug Des. 1996, 11, 15; Iida, H., et al., Recent Res. Dev. Synth. Org. Chem. 1998, 1, 17; Jia, G., et al., Heterocycl. Commun. 1998, 4, 557; Jia, G., et al., Chem. Commun. 1999, 119; Tao, Z.-F., et al., Angew. Chem., Int. Ed. 1999, 38, 650; Tao, Z.-F., et al., J. Am. Chem. Soc. 1999, 121, 4961; Tao, Z.-F., et al., J. Am. Chem. Soc. 1999, 121, 4961; Amishiro, N., et al., Chem. Pharm. Bull. 1999, 47, 1393; Tao, Z.-F., et al., J. Am. Chem. Soc. 2000, 122, 1602; Chang, A. Y., et al., J. Am. Chem. Soc. 2000, 122, 4856; Atwell, G. J., et al., J. Med. Chem. 1999, 42, 3400; and Baraldi, P. G., et al., J. Med. Chem. 2001, 44, 2536).

What is needed is to design and synthesize a complete series of CBI analogues of CC-1065 and the duocarmycins having dimeric monocyclic, bicyclic, and tricyclic heteroaromatics substituents.

What is needed is to characterize the effects of these dimeric monocyclic, bicyclic, and tricyclic heteroaromatics substituents upon the activity of the resultant CBI analogues of CC-1065 and the duocarmycins so as to demonstrate that the contribution of these substituents within DNA binding domain goes beyond simply providing AT-rich noncovalent binding affinity and supports an additional primary role with respect to the catalytic activity of these compounds.

### SUMMARY

The solution phase parallel synthesis and evaluation of a library of 132 CBI analogues of CC-1065 and the duocarmycins containing dimeric monocyclic, bicyclic, and tricyclic (hetero)aromatic replacements for the DNA binding domain are described. The library was then employed to characterize the structural requirements for potent cytotoxic activity and DNA alkylation efficiency. Key analogues within the library displayed enhanced activity, the range of which span a magnitude of ≧10,000-fold. Combined with related studies, these results highlight that role of the DNA binding domain goes beyond simply providing DNA binding selectivity and affinity (10-100-fold enhancement in properties), consistent with the proposal that it contributes significantly to catalysis of the DNA alkylation reaction accounting for as much as an additional 1000-fold enhancement in properties.

Because of its synthetic accessibility, its potency and efficacy which matches or exceeds that of the CC-1065 MeCPI alkylation subunit, and the extensive documentation of the biological properties of its derivatives, the library was assembled using the seco precursor 4 to the (+)-1,2,9,9a-tetrahydrocyclopropa[c]benz[e]indole-4-one (CBI) alkylation subunit (FIG. 2) (Boger, D. L., et al., J. Am. Chem. Soc. 1989, 111, 6461; Boger, D. L., et al., J. Org. Chem. 1990, 55, 5823; Boger, D. L., et al., Tetrahedron Lett. 1990, 31, 793; Boger, D. L., et al., J. Org. Chem. 1992, 57, 2873; Boger, D. L., et al., J. Am. Chem. Soc. 1994, 116, 7996; Boger, D. L., et al., J. Org. Chem. 1995, 60, 1271; Boger, D. L., et al., Synlett 1997, 515; Boger, D. L., et al., Tetrahedron Lett. 1998, 39, 2227; Boger, D. L., et al., Synthesis 1999, 1505; Boger, D. L., et al., Bioorg. Med. Chem. 1995, 3, 1429; Boger, D. L., et al., Bioorg. Med. Chem. 1995, 3, 761; and Boger, D. L., et al., J. Am. Chem. Soc. 1992, 114, 5487). To date, no distinctions between the seco-CBI and CBI derivatives have been detected in a range of in vitro and in vivo assays in accordance with past studies of all such alkylation subunits (Boger, D. L., et al., Chem. Rev. 1997, 97, 787), indicating that in situ spirocyclization is not rate determining or property limiting.

One aspect of the invention is directed to a compound represented by either of the following two structures: 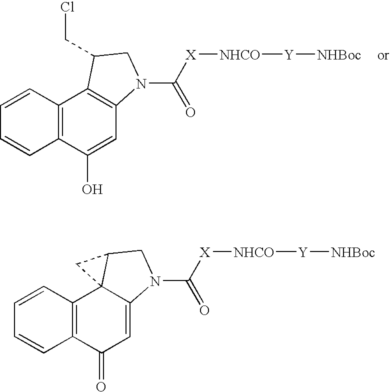
In the above structure, —C(O)XNH— is selected from one of the biradicals represented by the following structures: 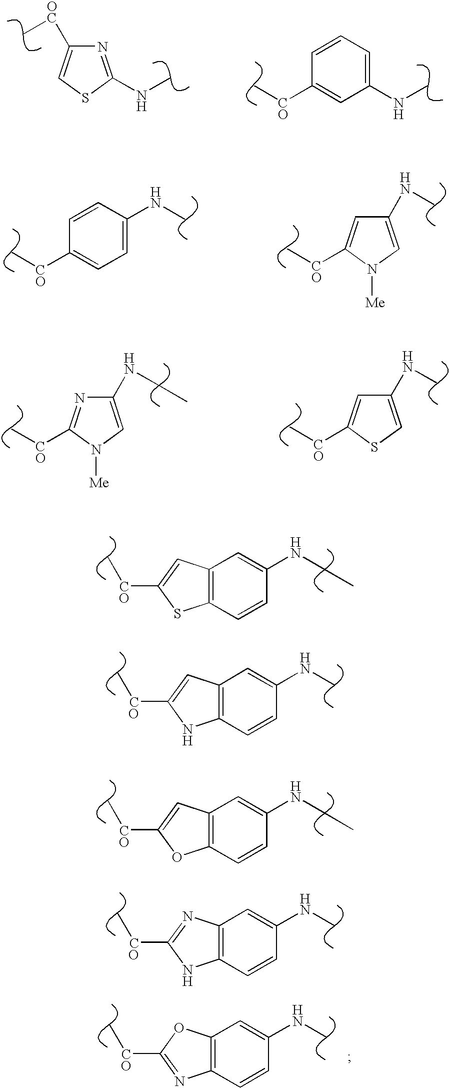
Similarly, —C(O)YNH— is selected from one of the diradicals represented by the following structures: 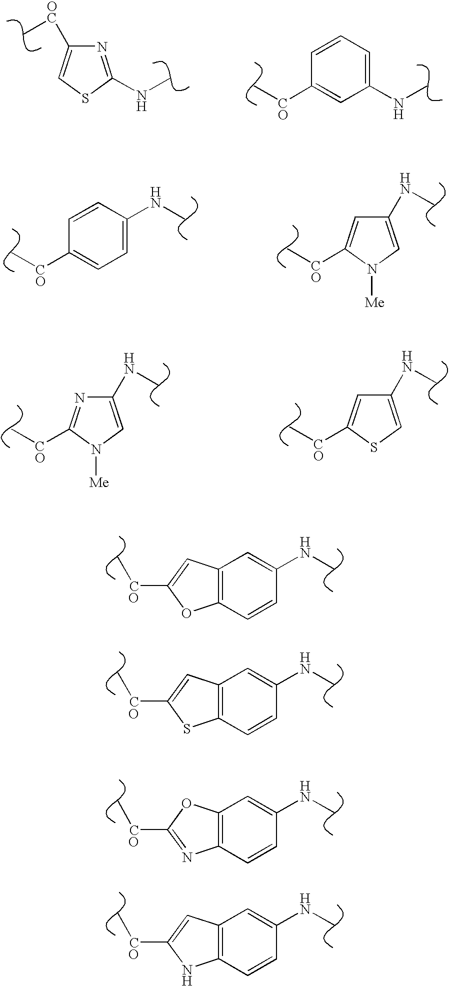
However, there is a proviso that if —C(O)XNH— is either 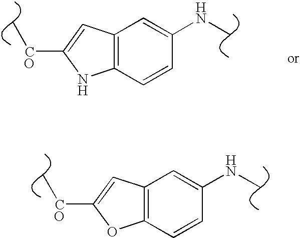
then —C(O)YNH— can not be any of 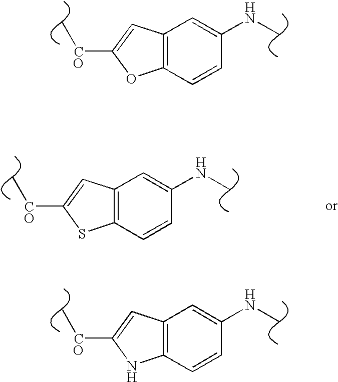
In a preferred mode of this invention, —C(O)XNH— is selected from the group of biradicals consisting of: 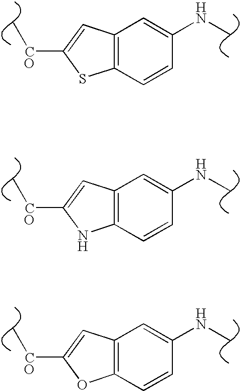
Also, in each instance, the -Boc protecting/blocking group on the terminal amino group may be replaced by a functionally equivalent protecting/blocking group.

Another aspect of the invention is directed to a compound represented by the following structures: 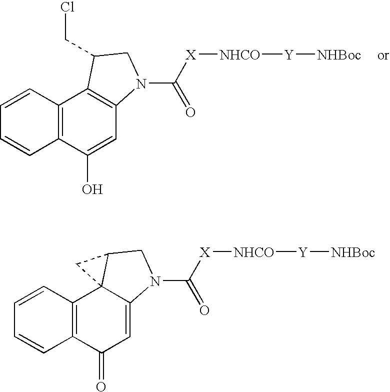
In the above structure, —C(O)XN— is represented by the following diradical: 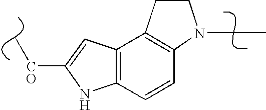
On the other hand, —C(O)YNH— is selected from the diradicals represented by the following structures: 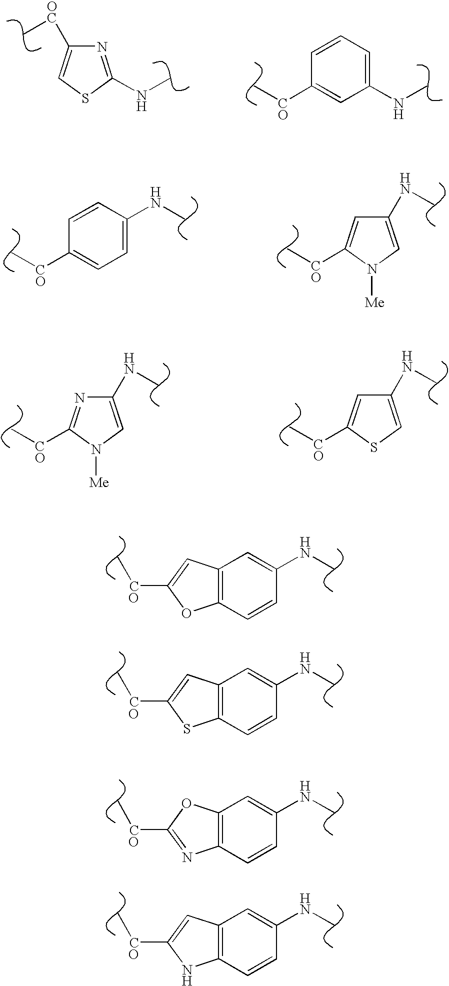
In each instance, the -Boc protecting/blocking group on the terminal amino group may be replaced by a functionally equivalent protecting/blocking group.

Another aspect of the invention is a compound represented by the following structure: 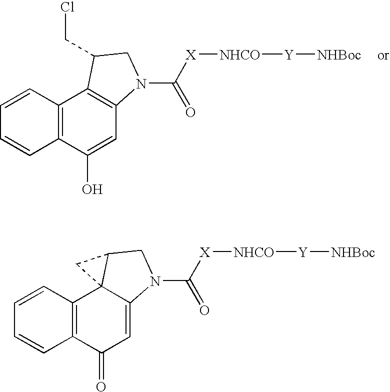
In the above structure, —C(O)XNH— is selected from the diradicals represented by the following structures: 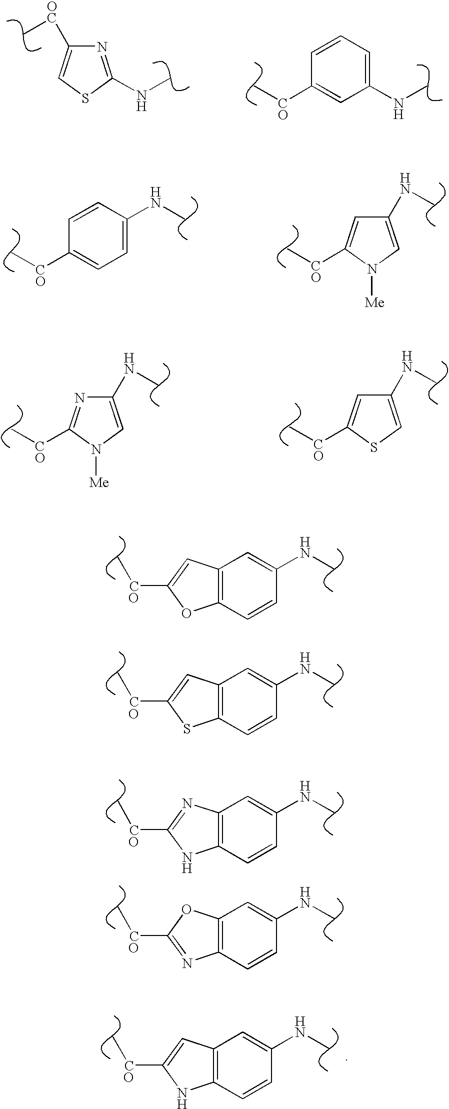
On the other hand, —C(O)YN— is represented by the following diradical: 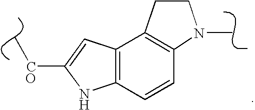
In each instance, the -Boc protecting/blocking group on the terminal amino group may be replaced by a functionally equivalent protecting/blocking group.

Another aspect of the invention is directed to a process for killing a cancer cell. The process employs the step of contacting the cancer cell with a composition having a cytotoxic concentration of one or more of the compounds described above. The cytotoxic concentration of the composition is cytotoxic with respect to the cancer cell.

The parallel synthesis of 132 CBI analogues of CC-1065 and the duocarmycins, employed herein, utilizes the solution-phase technology of acid-base liquid-liquid extraction for their isolation and purification. The 132 analogues constitute a systematic study of the DNA binding domain with the incorporation of dimers composed of monocyclic, bicyclic, and tricyclic (hetero)aromatic subunits. From their examination, clear trends in cytotoxic potency and DNA alkylation efficiency emerge highlighting the principle importance of the first attached DNA binding subunit (X subunit): tricyclic>bicyclic>monocyclic (hetero)aromatic subunits. Notably the trends observed in the cytotoxic potencies parallel those observed in the relative efficiencies of DNA alkylation. It is disclosed herein that these trends represent the partitioning of the role of the DNA binding subunit(s) into two distinct contributions, viz., 1.) a first contribution derived from an increase in DNA binding selectivity and affinity which leads to property enhancements of 10-100-fold and is embodied in the monocyclic group 1 series; and 2.) a second contribution, additionally and effectively embodied in the bicyclic and tricyclic heteroaromatic subunits, provides additional enhancements of 100-1000-fold with respect to catalysis of the DNA alkylation reaction. The total overall enhancement can exceed 25,000-fold. Aside from the significance of these observations in the design of future CC-1065/duocarmycin analogues, their significance to the design of hybrid structures containing the CC-1065/duocarmycin alkylation subunit should not be underestimated. Those that lack an attached bicyclic or tricyclic X subunit, i.e. duocarmycin/distamycin hybrids, can be expected to be intrinsically poor or slow DNA alkylating agents.

### BRIEF DESCRIPTION OF FIGURES

FIG. 1 illustrates the structures of CC-1065 (1) and the duocarmycins (2 and 3).

FIG. 2 illustrates structures for various alkylating subunits of the anti-tumor antibiotics.

FIG. 3 illustrates structures for the various subunits that make up the library.

FIG. 4 is a scheme which illustrates the steps required to synthesize the 132 members of the library.

FIG. 5 illustrates a chart which shows the evaluation of the CBI-based analogues in a cellular functional assay for L1210 cytotoxic activity revealed a clear relationship between the potency of the agents and the structure of the DNA binding domain.

FIG. 6 illustrates the structures of the series of agents 21, containing an indole ring, 22, containing a benzoxazole ring, and 23, which contains a benzimidazole ring.

FIG. 7 illustrates the structures of compound 24, 25, 26, 27 and 28 which were compared on the basis of their DNA alkylation properties.

FIG. 8 illustrates a polyacrylamide gel electrophoresis (PAGE) which has the Sanger dideoxynucleotide sequencing standards and shows evidence of DNA strand cleavage by the reagents listed.

### DETAILED DESCRIPTION

The parallel synthesis of 132 CBI analogues of CC-1065 and the duocarmycins, employed herein, utilizes the solution-phase technology of acid-base liquid-liquid extraction for their isolation and purification. The 132 analogues constitute a systematic study of the DNA binding domain with the incorporation of dimers composed of monocyclic, bicyclic, and tricyclic (hetero)aromatic subunits. From their examination, clear trends in cytotoxic potency and DNA alkylation efficiency emerge highlighting the principle importance of the first attached DNA binding subunit (X subunit): tricyclic>bicyclic>monocyclic (hetero)aromatic subunits. Notably the trends observed in the cytotoxic potencies parallel those observed in the relative efficiencies of DNA alkylation. It is disclosed herein that these trends represent the partitioning of the role of the DNA binding subunit(s) into two distinct contributions, viz., 1.) a first contribution derived from an increase in DNA binding selectivity and affinity which leads to property enhancements of 10-100-fold and is embodied in the monocyclic group 1 series; and 2.) a second contribution, additionally and effectively embodied in the bicyclic and tricyclic heteroaromatic subunits, provides additional enhancements of 100-1000-fold with respect to catalysis of the DNA alkylation reaction. The total overall enhancement can exceed 25,000-fold. Aside from the significance of these observations in the design of future CC-1065/duocarmycin analogues, their significance to the design of hybrid structures containing the CC-1065/duocarmycin alkylation subunit should not be underestimated. Those that lack an attached bicyclic or tricyclic X subunit, i.e. duocarmycin/distamycin hybrids, can be expected to be intrinsically poor or slow DNA alkylating agents.

Synthesis of the 132-Membered Library:

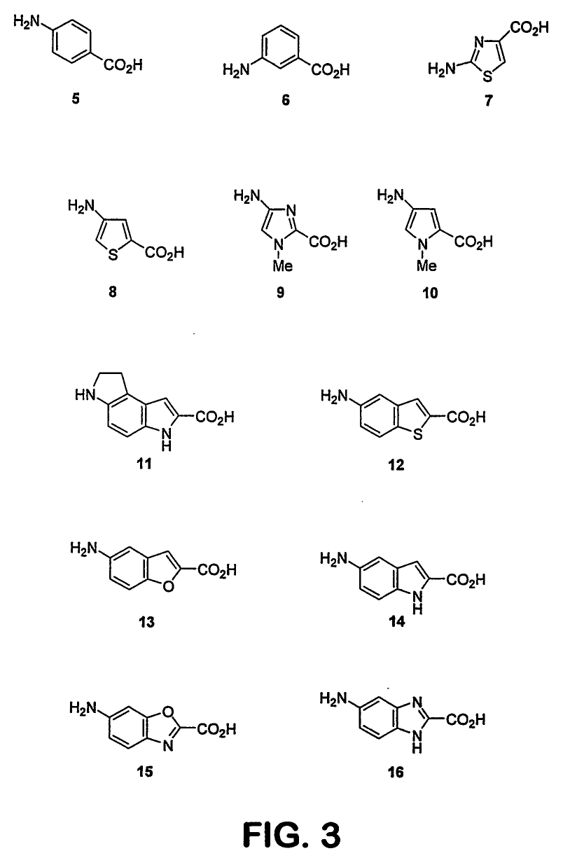

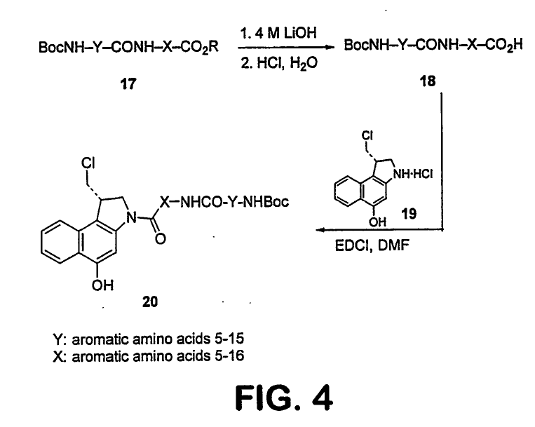

A recent study by Boger et al., detailed the parallel synthesis of a 132-membered library of heteroaromatic dimers related to the structures of distamycin and CC-1065 (Boger, D. L., et al., Am. Chem. Soc. 2000, 122, 6382). This study included the monocyclic, bicyclic, and tricyclic (hetero)aromatic amino acids 5-16 (FIG. 3), which have been explored in the examination of these two natural products. The 132 dimers composed of these subunits were assembled by parallel synthesis through formation of the linking amide enlisting a simple acid-base liquid-liquid extraction protocol for isolation and purification. Each of the 132 dimers were fully characterized (Boger, D. L., et al., Am. Chem. Soc. 2000, 122, 6382) and used for the formation of the library of CBI analogues. Dimers employing uncharged protecting groups other than Boc for blocking the terminal amino group may also be employed for making the seco-CBI analogues and CBI analogues of CC-1065 and the duocarmycins with substantially equivalent activity, i.e., functional equivalents may be employed and are encompassed within the scope of the invention. Each dimer was saponified by treatment with LiOH (4 M aqueous solution in dioxane-water 4:1 for 12 hours, 25° C.) to afford the lithium salts of the carboxylic acids (FIG. 4). Hydrolysis of the compounds that possessed the 3-amino-1-methylpyrrole-5-carboxylate (10) or 6-aminoindole-2-carboxylate (14) subunits at the C-terminus was slower and the reactions were conducted at 40° C. Acidifying of the aqueous Li-salt solutions gave the free carboxylic acids 18 that were used for the subsequent couplings without further purification. Notably, the dimers with the 6-aminobenzoxazole-2-carboxylate (15) and 6-aminobenzimidazole-2-carboxylate (16) subunits at the C-terminus, which are prone to decarboxylation (Boger, D. L., et al., Am. Chem. Soc. 2000, 122, 6382), were sufficiently stable for use in the next conversion. After deprotection of 4 (4 M HCl-EtOAc, 25° C., 45 min), the resulting hydrochloride 19 was coupled with the dimer carboxylic acids using 1-[3-(dimethylamino)propyl]-3-ethylcarbodiimide hydrochloride (EDCl) to provide 20. Simple acid/base extraction and purification with aqueous 3 N HCl/saturated aqueous Na₂CO₃ yielded each analogue sufficiently pure for direct assay.

Each of the seco-CBI analogues of CC-1065 and the duocarmycins may be easily converted to the corresponding CBI analogue of CC-1065 and the duocarmycins in the presence of base, e.g., DBU (Boger, D. L., et al., Chem. Rev. 1997, 97, 787).

Cytotoxic Activity:

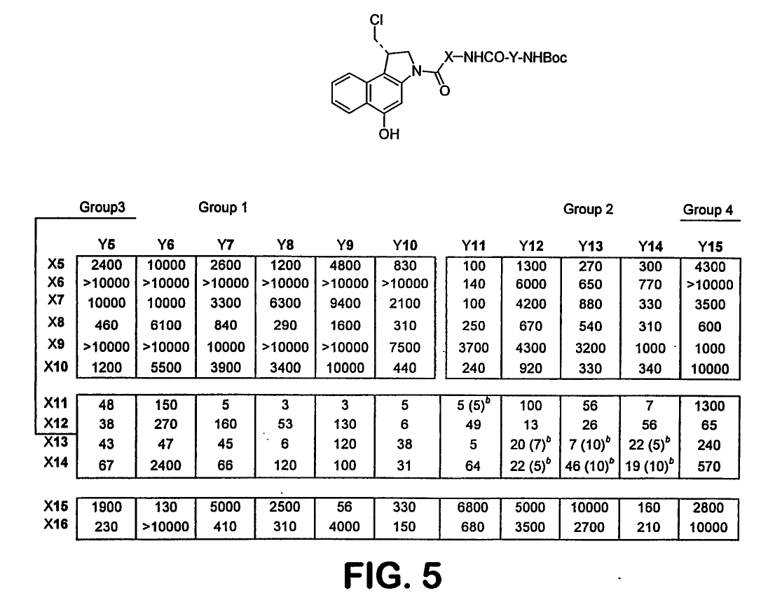

Evaluation of the CBI-based analogues in a cellular functional assay for L1210 cytotoxic activity revealed a clear relationship between the potency of the agents and the structure of the DNA binding domain (FIG. 5). For comparison, the L1210 IC₅₀ for (+)-N-Boc-CBI, which lacks an attached DNA binding domain, is 80 nM (80,000 pM). With a few exceptions, all group 1 compounds containing two monocyclic subunits (5-10 in positions X and Y) exhibited IC₅₀ values between 1-10 nM or higher indicating an increase in potency of approximately 10-fold relative to N-Boc-CBI. The exception is the thiophene subunit 8, which when incorporated as the X subunit adjacent to the DNA alkylation subunit, exhibited slightly greater potency. The best in this series were X8-Y8 (290 pM, 275-fold enhancement) and X8-Y10 (310 pM, 260-fold enhancement). Notably, the distamycin/netropsin dipyrrole was also effective with X10-Y10 (440 pM) exhibiting a 180-fold enhancement. Nonetheless, even the best in this series exhibited a modest ca. 100-fold enhancement over (+)-N-Boc-CBI and typically it constituted a much more modest 10-100-fold enhancement. Within the group 1 dimers, it is also interesting that the 4-aminobenzoic acid subunit (5, X group) compares favorably with the distamycin N-methyl-4-aminopyrrole-2-carboxylic acid subunit (10) providing IC₅₀'s that are within 2-3 fold of one another, whereas the 3-aminobenzoic acid subunit (6) or the imidazole (9) are not effective.

An analogous level of potency (10-100-fold enhancement) was observed with the group 2 monocyclic heteroaromatics (X group) when they were coupled to a terminal bicyclic heteroaromatic subunit (12-15) and a slightly greater enhancement was observed when the Y subunit was tricyclic (11). Notably, none of the compounds in this group 1 or group 2 series drop below IC₅₀'s of 100 pM or approach the potency of the natural products.

In contrast to these analogues, the group 3 dimers with the bicyclic and tricyclic subunits 11-14 bound directly to the DNA alkylation subunit constitute an array of substances with much greater cytotoxic potency. The potency enhancement observed with the analogues containing a bicyclic or tricyclic X subunit linked directly to the alkylation subunit (the group 3, X11-14 subunits) typically range from 27,000-1000 (IC₅₀=3-80 pM) relative to N-Boc-CBI. This is also roughly a 100-1000-fold enhancement over the monocyclic X subunits. All compounds in the library with IC₅₀'s below 10_pM can be found in this collection and two-thirds of them contain the tricyclic CDPI subunit (11) in this key position, i.e., X11-Y7 (5 pM), X11-Y8 (3 pM), X11-Y9 (3 pM), X11-Y10 (5 pM), X11-Y11 (5 pM) and X11-Y14 (7 pM). In this regard, it seems advantageous to have an five-membered heterocycle in Y position with CDPI (11) in the X position.

The proposal of binding-induced catalysis for DNA alkylation by CC-1065 (1) and related compounds in which the shape and size of the substituent directly bound to the vinylogous amide makes a major contribution to the properties is supported by the trends within the library. Compounds having the extended subunits 11-14 in the X position and smaller subunits 7-10 in Y position show higher potency (typically 10-100-fold) than the corresponding compounds with inverted sequences. Since the bound agent is forced to follow the inherent helical twist of the minor groove, the helical rise induced in the molecule can only be adjusted by twisting the linking amide that connects the noncovalent binding subunit with the vinylogous amide of the alkylation subunit. The more extended the subunit, the greater the twist in the linking amide resulting in an increased activation of the agent. The lower cytotoxicity exhibited by analogues made from dimers consisting of the five-membered heterocycles 5-10 is also consistent with this explanation. Although these subunits are well known as minor groove binding constituents of distamycin, netropsin, and lexitropsins, they lack the rigid length that the fused aromatic heterocycles possess.

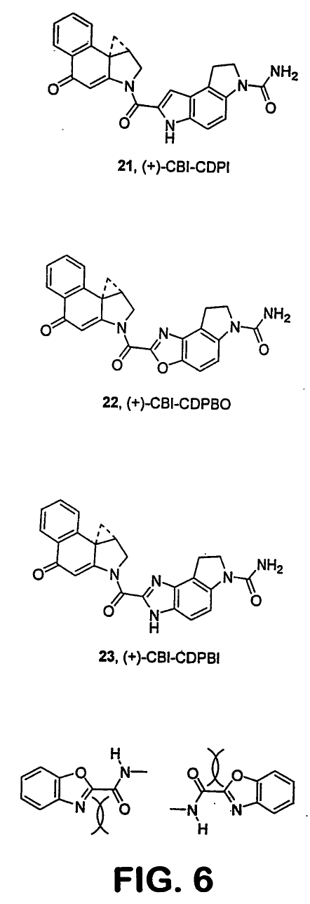

Compared to the analogues possessing benzothiophene (12), benzofuran (13) or indole (14) at the X-position of the dimer, agents containing benzoxazole (15) or benzimidazole (16) in this position (group 4) exhibit a considerable decrease in potency, up to 130-fold for X15-Y13. Similar observations have been made in a previous study concerning deep-seated modifications of the DNA binding subunit of CC-1065 (FIG. 6) (Boger, D. L., et al., Bioorg. Med. Chem. 1995, 3, 1429; Boger, D. L., et al., Bioorg. Med. Chem. 1995, 3, 761). The introduction of an additional heteroatom in the carboxylate bearing aromatic ring of (+)-CBI-CDPI (21) led to a 40-fold decrease in cytotoxic activity and an analogous decrease in the DNA alkylation efficiency observed with (+)-CBI-CDPBO (22) and (+)-CBI-CDPBI (23), but no alteration in the alkylation selectivity compared to the parent compound. This was attributed to the destabilizing electrostatic interactions between the amide carbonyl lone pair and the heteroatom lone pairs present when the amide carbonyl adopts either of the in plane conjugated conformations (FIG. 6). This interaction results in a twist of the C-terminal bicyclic aromatic ring out of the plane defined by the carboxamide precluding preferential adoption of a near planar conformation that facilitates minor groove binding.

DNA Alkylation Efficiency and Selectivity:

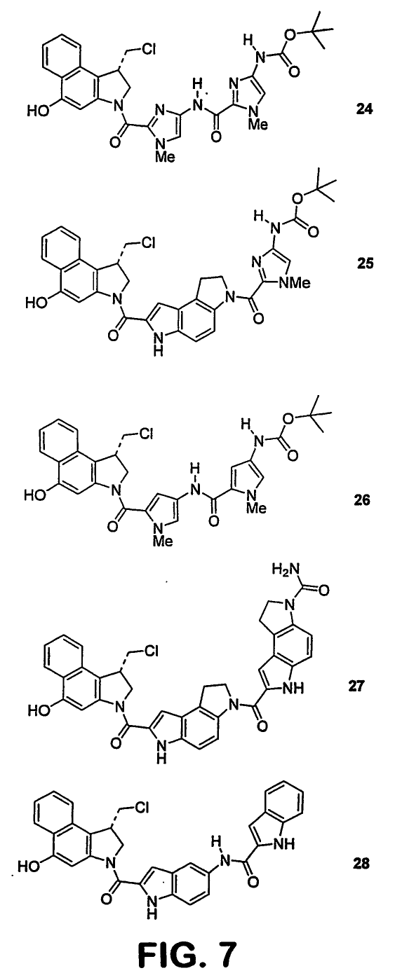

The DNA alkylation properties of the compounds including those of CBI-X9-Y9 (24), CBI-X11-Y9 (25) and CBI-X10-Y10 (26) (FIG. 7) were examined within a 150 base-pair segment of duplex DNA and compared with (+)-duocarmycin SA (2), (+)-CBI-CDPI₂ (27) and (+)-CBI-indole₂ (28). One clone of phage M13mp10 was selected for the study that contained the SV40 nucleosomal DNA insert w794 (nucleotide no. 5238-138) (Ambrose, C., et al., J. Mol. Biol. 1989, 210, 255). The alkylation site identification and the assessment of the relative selectivity among the available sites was obtained by thermally-induced strand cleavage of the singly 5′ end-labeled duplex DNA after exposure to the agents. After treatment of the end-labeled duplex DNA with a range of agent concentrations, the unbound agent was removed by EtOH precipitation of the DNA. Redissolution of the DNA in aqueous buffer, thermolysis (100° C., 30 min) to induce strand cleavage at the sites of DNA alkylation, denaturing high resolution polyacrylamide gel electrophoresis (PAGE) adjacent to Sanger dideoxynucleotide sequencing standards, and autoradiography led to identification of the DNA cleavage and alkylation sites (Boger, D. L., et al., Tetrahedron 1991, 47, 2661).

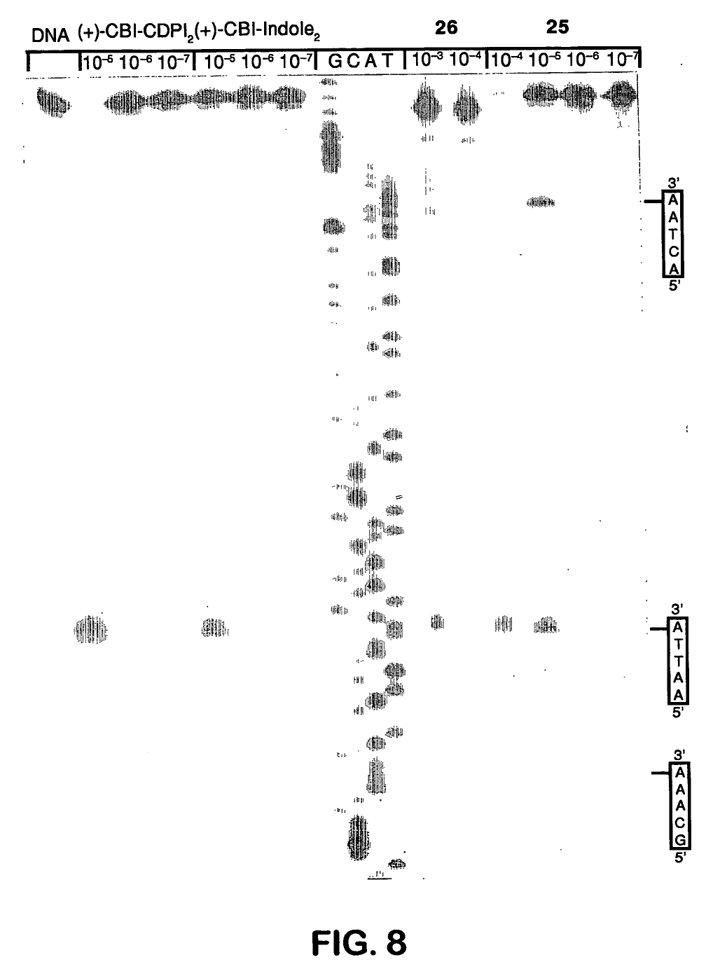

Representative of the comparisons made and the trends observed, the analogues 25 and 26 were found to detectably alkylate DNA at 10⁻⁵-10⁻⁶ M and 10⁻³ M, respectively, whereas alkylation by 24 (not shown) could not be observed even at 10⁻³ M (FIG. 8). Throughout the comparisons, the relative DNA alkylation efficiencies were found to parallel the cytotoxic potencies of the compounds. Thus, the 100-fold lower cytotoxicity of 26 compared to 25 is also reflected in the 100-1000-fold lower alkylation efficiency of 26. This behavior is dramatic with 26 being only 10-100 fold more effective than N-Boc-CBI which alkylates DNA at 10⁻¹-10⁻² M under comparable reaction conditions albeit with a reduced selectivity. Thus, while the dipyrrole binding subunit does enhance the DNA alkylation efficiency and selectivity relative to N-Boc-CBI, it is also substantially less effective (100-1000-fold) than the compounds containing bicyclic or tricyclic X groups. The significance of those observations should not be underestimated and suggest that hybrid agents composed of the CC-1065/duocarmycin related alkylation subunits and distamycin/netropsin DNA binding subunits are intrinsically poor DNA alkylating agents.

Notably, no alterations in the DNA alkylation selectivities were observed despite the changes in the DNA binding domain except for the minor differences noted before. Thus, although the efficiency of DNA alkylations were altered greatly, the selectivity was not. Within the w794 segment of DNA, a major alkylation site (5′-AATTA-3′) and two minor sites (5′-ACTAA-3′, 5′-GCAAA-3′) are observed with the natural enantiomers. The relative extent to which alkylation at the minor sites is observed is dependent on the overall size (length) of the agent and the extent of DNA alkylation. For example, neither 27 or 28 alkylate the minor 5′-ACTAA-3′ site to a significant extent while the shorter agent 25, like 21, does (Boger, D. L., et al., J. Am. Chem. Soc. 1992, 114, 5487). In addition, the minor 5′-GCAAA-3′ site only appears on the gel after near complete consumption of the end-labeled DNA indicative of extensive, multiple DNA alkylations resulting in cleavage to shorter fragments of DNA. Other than these minor distinctions in the DNA alkylation selectivity which have been noted in prior studies of CBI derivatives (Boger, D. L., et al., J. Am. Chem. Soc. 1992, 114, 5487), no significant changes were observed with variations in the DNA binding subunits. Thus, while it may appear reasonable to suggest that the alkylation of the 5′-ACTAA-3′ site by 25 is a result of imidazole H-bonding to the intervening GC base-pair, the identical behavior of (+)-CBI-CDPI (21), which lacks this subunit, suggests it is simply a natural consequence of a shorter agent binding and alkylating DNA within a shorter AT-rich sequence (Boger, D. L., et al., J. Am. Chem. Soc. 1992, 114, 5487) It is important to recognize that the X subunit C5 substituent contributes significantly to the rate and efficiency of DNA alkylation and cytotoxic activity presumably by extending the rigid length of the X subunit. In studies of analogues which lack a third Y subunit, the presence of a C5 substituent on the bicyclic X subunit substantially (10-1000-fold) enhances the properties providing analogues comparable in cytotoxic potency and DNA alkylation efficiency to the best analogues detailed herein. See the following: Boger, D. L., et al., J. Am. Chem. Soc. 1997, 119, 4977; Boger, D. L., et al., J. Am. Chem. Soc. 1997, 119, 4987; and Boger, D. L., et al., Bioorg. Med. Chem. Lett. 2001, 11, 2021.

The parallel synthesis of 132 CBI analogues of CC-1065 and the duocarmycins was described utilizing the solution-phase technology of acid-base liquid-liquid extraction for their isolation and purification. The 132 analogues constitute a systematic study of the DNA binding domain with the incorporation of dimers composed of monocyclic, bicyclic, and tricyclic (hetero)aromatic subunits. From their examination, clear trends in cytotoxic potency and DNA alkylation efficiency emerge highlighting the principle importance of the first attached DNA binding subunit (X subunit): tricyclic>bicyclic>monocyclic (hetero)aromatic subunits. Notably the trends observed in the cytotoxic potencies parallel those observed in the relative efficiencieś of DNA alkylation. It is disclosed herein that these trends represent the partitioning of the role of the DNA binding subunit(s) into two distinct contributions, viz., 1.) a first contribution derived from an increase in DNA binding selectivity and affinity which leads to property enhancements of 10-100-fold and is embodied in the monocyclic group 1 series; and 2.) a second contribution, additionally and effectively embodied in the bicyclic and tricyclic heteroaromatic subunits, provides additional enhancements of 100-1000-fold with respect to catalysis of the DNA alkylation reaction. The total overall enhancement can exceed 25,000-fold. Aside from the significance of these observations in the design of future CC-1065/duocarmycin analogues, their significance to the design of hybrid structures containing the CC-1065/duocarmycin alkylation subunit should not be underestimated. Those that lack an attached bicyclic or tricyclic X subunit, i.e. duocarmycin/distamycin hybrids, can be expected to be intrinsically poor or slow DNA alkylating agents.

General Procedure for Preparation of the CBI Analogues:

A solution of the dimer ester 17 (20 μmol) (Boger, D. L., et al., Am. Chem. Soc. 2000, 122, 6382) in dioxane-water (4:1, 250-300 μL) was treated with aqueous LiOH (4 M, 20 μL) and the mixture was stirred for 12 hours at 20-25° C. After lyophilization, the crude material was dissolved in water (500 μL), treated with aqueous HCl (3 M, 100 μL) and the precipitate collected by centrifugation. Decantation and lyophilization of the residue from water (500 μL) yielded material (18) that was sufficiently pure for the subsequent coupling. A sample of 4 (1 mg, 3 μmol) (Boger, D. L., et al., J. Am. Chem. Soc. 1989, 111, 6461; Boger, D. L., et al., J. Org. Chem. 1990, 55, 5823; Boger, D. L., et al., Tetrahedron Lett. 1990, 31, 793; Boger, D. L., et al., J. Org. Chem. 1992, 57, 2873; Boger, D. L., et al., J. Am. Chem. Soc. 1994, 116, 7996; Boger, D. L., et al., J. Org. Chem. 1995, 60, 1271; Boger, D. L., et al., Synlett 1997, 515; Boger, D. L., et al., Tetrahedron Lett. 1998, 39, 2227; Boger, D. L., et al., Synthesis 1999, 1505) was treated for 45 min with HCl-EtOAc (4 M, 300 μL). After evaporation of the solvent under a steady stream of N₂, the residue was dried in vacuo. The crude material was dissolved in DMF (40 μL) together with EDCl (9 μmol, 1.7 mg) and 18 (4.5 μmol) and allowed to stand at 20-25° C. The reaction was quenched after 12 hours by adding saturated aqueous NaCl (400 μL). Isolation of the product was performed by extraction with EtOAc (4×600 μL), subsequent washing of the organic layer with aqueous 3 M aqueous HCl (4×400 μL), saturated aqueous Na₂CO₃ (4×400 μL) and saturated aqueous NaCl (1×400 μL). The combined organic layers were dried (Na₂SO₄), and concentrated to afford the CBI analogue in yields between 30% and 97%.

The diagonal elements of the library and additional selected members were characterized by ¹H NMR and HRMALDI-FTMS.
1-(Chloromethyl)-5-hydroxy-3-{4-[4-(tert-Butoxycarbonylamino)benzoyl]aminobenzoyl}-1,2-dihydrobenzo[e]indole(seco-CBI-X5-Y5): (0.99 mg, 58%); HRMALDI-FTMS (DHB) m/z 572.1943 (C₃₂H₃₀CIN₃O₅+H⁺ requires 572.1952).
1-(Chloromethyl)-5-hydroxy-3-{3-[3-(tert-Butoxycarbonylamino)benzoyl]aminobenzoyl}-1,2-dihydrobenzo[e]indole (seco-CBI-X6-Y6): (0.95 mg, 55%); HRMALDI-FTMS (DHB) m/z 558.1995 (C₃₂H₃₀CIN₃O₅−HCl+Na⁺ requires 558.2005).
1-(Chloromethyl)-5-hydroxy-3-{[2-[2-(tert-Butoxycarbonylamino-1,3-thiazol-4-yl)carbonyl]amino-1,3-thiazol-4-yl]carbonyl}-1,2-dihydrobenzo[e]indole (seco-CBI-X7-Y7): (1.12 mg, 64%); HRMALDI-FTMS (DHB) m/z 608.0814 (C₂₆H₂₄CIN₅O₅S₂+Na⁺ requires 608.0805).
1-(Chloromethyl)-5-hydroxy-3-{[2-[4-(tert-Butoxycarbonylamino)-1-methylimidazol-2-yl)-carbonyl]amino-1,3-thiazol-4-yl]carbonyl}-1,2-dihydrobenzo[e]indole (seco-CBI-X7-Y9): (1.10 mg, 63%); HRMALDI-FTMS (DHB) m/z 583.1519 (C₂₇H₂₇CIN₆O₅S+H⁺ requires 583.1525).
1-(Chloromethyl)-5-hydroxy-3-{[2-[5-(tert-Butoxycarbonylaminobenzofuran-2-yl)carbonyl]amino-1,3-thiazol-4-yl]carbonyl}-1,2-dihydrobenzo[e]indole (seco-CBI-X7-Y13): (1.00 mg, 54%); HRMALDI-FTMS (DHB) m/z 641.1215 (C₃₁H₂₇CIN₄O₆S+Na⁺ requires 641.1232).
1-(Chloromethyl)-5-hydroxy-3-{[4-[4-(tert-Butoxycarbonylaminothiophen-2-yl)carbonyl]aminothiophen-2-yl]carbonyl}-1,2-dihydrobenzo[e]indole (seco-CBI-X8-Y8): (1.51 mg, 86%); HRMALDI-FTMS (DHB) m/z 570.1118 (C₂₈H₂₆CIN₃O₅S₂−HCl+Na⁺ requires 570.1133).
1-(Chloromethyl)-5-hydroxy-3-{[4-[4-(tert-Butoxycarbonylamino)-1-methylimidazol-2-yl)-carbonyl]amino-1-methylimidazol-2-yl]carbonyl}-1,2-dihydrobenzo[e]indole (seco-CBI-X9-Y9): (1.48 mg, 85%); HRMALDI-FTMS (DHB) m/z 580.2060 (C₂₈H₃₀CIN₇O₅+H⁺ requires 580.2075).
1-(Chloromethyl)-5-hydroxy-3-{[4-[4-(tert-Butoxycarbonylamino)-1-methylpyrrol-2-yl)carbonyl]amino-1-methylpyrrol-2-yl]carbonyl}-1,2-dihydrobenzo[e]indole (seco-CBI-X10-Y10): (1.18 mg, 68%); HRMALDI-FTMS (DHB) m/z 564.2233 (C₃₀H₃₂CIN₅O₅−HCl+Na⁺ requires 564.2223).
1-(Chloromethyl)-5-hydroxy-3-{[3-[2-(tert-Butoxycarbonylamino-1,3-thiazol-4-yl) carbonyl]-1,2-dihydro(3H-pyrrolo[3,2-e]indol)-7-yl)carbonyl}-1,2-dihydrobenzo[e]indole (seco-CBI-X11-Y7): (1.23 mg, 64%); HRMALDI-FTMS (DHB) m/z 544.1195 (C₃₃H₃₀CIN₅O₅S−Boc+H⁺ requires 544.1205).
1-(Chloromethyl)-5-hydroxy-3-{[3-[4-(tert-Butoxycarbonylamino)-1-methylpyrrol-2-yl)-carbonyl]-1,2-dihydro(3H-pyrrolo[3,2-e]indol)-7-yl)carbonyl}-1,2-dihydrobenzo[e]indole (seco-CBI-X11-Y10): (1.19 mg, 62%); HRMALDI-FTMS (DHB) m/z 626.2377 (C₃₅H₃₄CIN₅O₅−HCl+Na⁺ requires 626.2374).
1-(Chloromethyl)-5-hydroxy-3-{[3-[3-(tert-Butoxycarbonyl)-1,2-dihydro(3H-pyrrolo[3,2-e]indol)-7-yl)carbonyl]-1,2-dihydro(3H-pyrrolo[3,2-e]indol)-7-yl)carbonyl}-1,2-dihydro-benzo[e]indole (seco-CBI-X11-Y11): (1.06 mg, 50%); HRMALDI-FTMS (DHB) m/z 702.2478 (C₄₀H₃₆CIN₅O₅+H⁺ requires 702.2478).
1-(Chloromethyl)-5-hydroxy-3-{[3-[5-(tert-Butoxycarbonylaminoindole-2-yl)carbonyl]-1,2-dihydro(3H-pyrrolo[3,2-e]indol)-7-yl)carbonyl}-1,2-dihydrobenzo [e]indole (seco-CBI-X11-Y14): (0.91 mg, 45%); HRMALDI-FTMS (DHB) m/z 676.2309 (C₃₈H₃₄CIN₅O₅+H⁺ requires 676.2321).
1-(Chloromethyl)-5-hydroxy-3-{5-[4-(tert-Butoxycarbonylamino)-1-methylpyrrol-2-yl)carbonyl]aminobenzothiophen-2-yl]carbonyl}-1,2-dihydrobenzo[e]indole (seco-CBI-X12-Y10): (1.05 mg, 57%); HRMALDI-FTMS (DHB) m/z 495.1504 (C₃₃H₃₁CIN₄O₅S−Boc−HCl+H⁺ requires 495.1491).
1-(Chloromethyl)-5-hydroxy-3-{[5-[5-(tert-Butoxycarbonylaminobenzothiophene-2-yl)carbonyl]aminobenzothiophene-2-yl]carbonyl}-1,2-dihydrobenzo[e]indole (seco-CBI-X12-Y12): (1.81 mg, 88%); HRMALDI-FTMS (DHB) m/z 684.1366 (C₃₆H₂₉CIN₃O₅S₂+H⁺ requires 684.1388).
1-(Chloromethyl)-5-hydroxy-3-{[5-[4-(tert-Butoxycarbonylamino)benzoyl]aminobenzo-furan-2-yl]carbonyl}-1,2-dihydrobenzo[e]indole (seco-CBI-X13-Y5): (1.78 mg, 97%); HRMALDI-FTMS (DHB) m/z 598.1946 (C₃₄H₃₀CIN₃O₆−HCl+Na⁺ requires 598.1949).
1-(Chloromethyl)-5-hydroxy-3-{[5-[4-(tert-Butoxycarbonylaminothiophen-2-yl)carbonyl]amino-benzofuran-2-yl]carbonyl}-1,2-dihydrobenzo[e]indole (seco-CBI-X13-Y8): (0.91 mg, 48%); HRMALDI-FTMS (DHB) m/z 517.0855 (C₃₂H₂₈CIN₃O₆S⁺−Boc requires 517.0863).
1-(Chloromethyl)-5-hydroxy-3-{[5-[5-(tert-Butoxycarbonylaminobenzofuran-2-yl)carbon-yl]aminobenzofuran-2-yl]carbonyl}-1,2-dihydrobenzo[e]indole (seco-CBI-X13-Y13): (1.32 mg, 67%); HRMALDI-FTMS (DHB) m/z 638.1883 (C₃₆H₃₀CIN₃O₇−HCl+Na⁺ requires 638.1903).
1-(Chloromethyl)-5-hydroxy-3-{[5-[5-(tert-Butoxycarbonylaminoindole-2-yl)carbon-yl]aminoindole-2-yl]carbonyl}-1,2-dihydrobenzo[e]indole (seco-CBI-X14-Y14): (1.39 mg, 71%); HRMALDI-FTMS (DHB) m/z 650.2149 (C₃₆H₃₂CIN₅O₅+H⁺ requires 650.2165).
1-(Chloromethyl)-5-hydroxy-3-{[6-[6-(tert-Butoxycarbonylaminobenzoxazole-2-yl)carbon-yl]aminobenzoxazole-2-yl]carbonyl}-1,2-dihydrobenzo[e]indole (seco-CBI-X15-Y15): (1.06 mg, 50%); HRMALDI-FTMS (DHB) m/z 653.1692 (C₃₄H₂₈CIN₅O₇+ requires 653.1671).
1-(Chloromethyl)-5-hydroxy-3-{[6-[4-(tert-Butoxycarbonylaminothiophene-2-yl)carbon-yl]aminobenzimidazole-2-yl]carbonyl}-1,2-dihydrobenzo[e]indole (seco-CBI-X16-Y8): (1.40 mg, 75%); HRMALDI-FTMS (DHB) m/z 618.1584 (C₃₁H₂₈CIN₅O₅S+H⁺ requires 618.1572).
   DNA Alkylation Studies: Selectivity and Efficiency.

The preparation of singly ³²P 5′ end-labeled double-stranded DNA, the agent binding studies, gel electrophoresis, and autoradiography were conducted according to procedures described in full detail elsewhere.²⁸ Eppendorf tubes containing the 5′ end-labeled DNA (9 μL) in TE buffer (10 mM Tris, 1 mM EDTA, pH 7.5) were treated with the agent in DMSO (1 μL at the specified concentration). The solution was mixed by vortexing and brief centrifugation and subsequently incubated at 25° C. for 24 hours. The covalently modified DNA was separated from the unbound agent by EtOH precipitation and resuspended in TE buffer (10 μL). The solution of DNA in an Eppendorf tube sealed with Parafilm was warmed at 100° C. for 30 min to introduce cleavage at the alkylation sites, allowed to cool to 25° C., and centrifuged. Formamide dye (0.03% xylene cyanol FF, 0.03% bromophenol blue, 8.7% Na₂EDTA 250 mM) was added (5 μL) to the supernatant. Prior to electrophoresis, the sample was denatured by warming at 100° C. for 5 min, placed in an ice bath, and centrifuged, and the supernatant (3 μL) was loaded directly onto the gel. Sanger dideoxynucleotide sequencing reactions were run as standards adjacent to the reaction samples. Polyacrylamide gel electrophoresis (PAGE) was run on an 8% sequencing gel under denaturing conditions (8 M urea) in TBE buffer (100 mM Tris, 100 mM boric acid, 0.2 mM Na₂EDTA) followed by autoradiography.

### DETAILED DESCRIPTION OF FIGURES

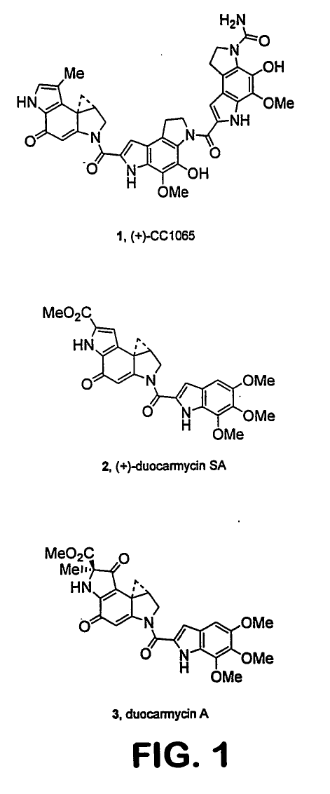

FIG. 1 shows the structures of CC-1065 (1) and the duocarmycins (2 and 3).

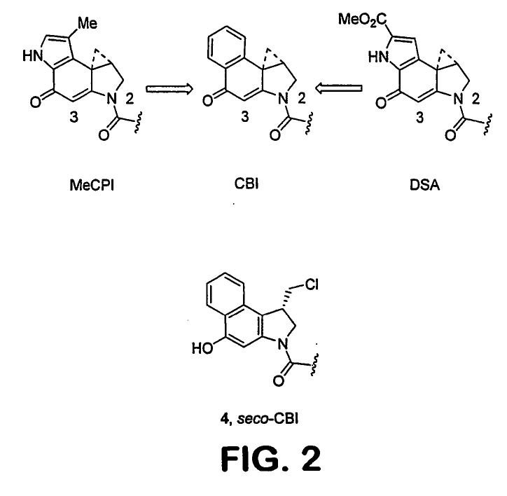

FIG. 2 shows the different structures of the various alkylating subunits of the anti-tumor antibiotics.

FIG. 3 gives the structures of the various subunits that make up the library.

FIG. 4 is a scheme which illustrates the steps required to synthesize the 132 members of the library. Each dimer was saponified by treatment with 4 M LiOH (aqueous solution in dioxane-water 4:1 for 12 h, 25° C.) to afford the lithium salts of the carboxylic acids. Acidification of the lithium salts gave the free carboxylic acids which could be coupled to the alkylating subunit 19.

FIG. 5 is a chart which shows the evaluation of the CBI-based analogues in a cellular functional assay for L1210 cytotoxic activity revealed a clear relationship between the potency of the agents and the structure of the DNA binding domain. For comparison, the L1210 IC₅₀ for (+)-N-Boc-CBI, which lacks an attached DNA binding domain, is 80 nM (80,000 pM).

FIG. 6 shows the structures of the series of agents 21, containing an indole ring, 22, containing a benzoxazole ring, and 23, which contains a benzimidazole ring. There is a decrease in potency of the DNA alkylating activity when another heteroatom is added to the carboxylate bearing aromatic ring. The introduction of an additional heteroatom in the carboxylate bearing aromatic ring of (+)-CBI-CDPI (21) led to a 40-fold decrease in cytotoxic activity and an analogous decrease in the DNA alkylation efficiency observed with (+)-CBI-CDPBO (22) and (+)-CBI-CDPBI (23), but no alteration in the alkylation selectivity compared to the parent compound. This is attributed to the destabilizing electrostatic interactions between the amide carbonyl lone pair and the heteroatom lone pairs present when the amide carbonyl adopts either of the in plane conjugated conformations as depicted in the last drawing.

FIG. 7 shows the structures of 24, 25, 26, 27 and 28 which were compared on the basis of their DNA alkylation properties. The first three compounds were examined with a 150 base-pair segment of duplex DNA and compared with duocarmycin SA (2), (+)-CBI-CDPI₂ (27) and (+)-CBI-indole₂ (28).

FIG. 8 is a polyacrylamide gel electrophoresis (PAGE) which has the Sanger dideoxynucleotide sequencing standards and shows evidence of DNA strand cleavage by the reagents listed. The analogues 25 and 26 were found to detectably alkylate DNA at 10⁻⁵-10⁻⁶ M and 10⁻³ M, respectively, whereas alkylation by 24 (not shown) could not be observed even at 10⁻³ M.

1. A compound represented by either of the following structures: 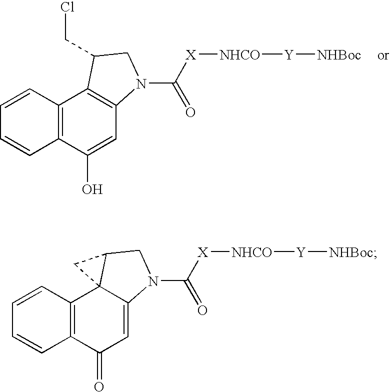
wherein —C(O)XNH— is selected from the group of biradicals consisting of: 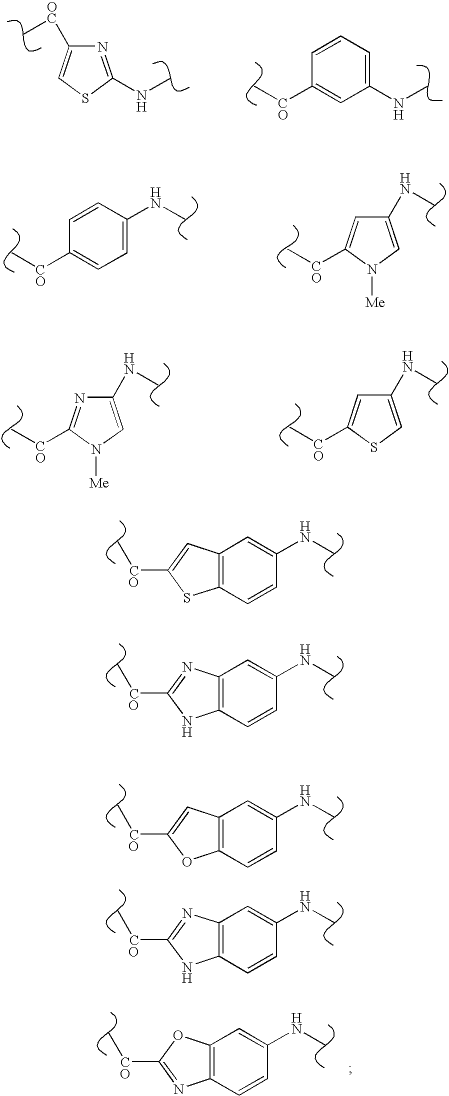
and —C(O)YNH— is selected from the group of diradicals consisting of: 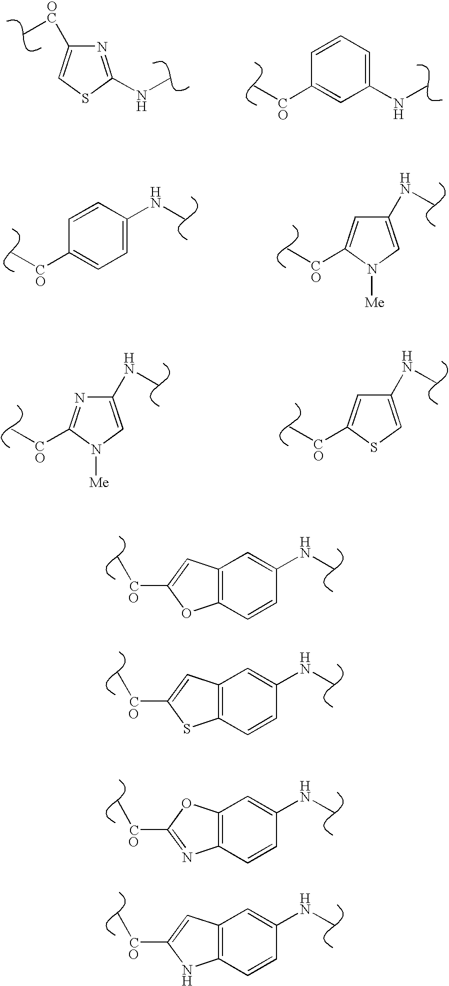
with a proviso that if —C(O)XNH— is either 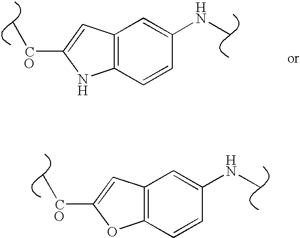
then —C(O)YNH— can not be any of

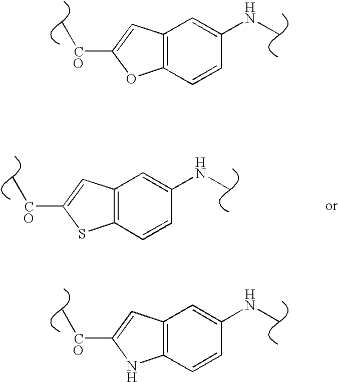

2. A compound according to claim 1 wherein:
—C(O)XNH— is selected from the group of biradicals consisting of:

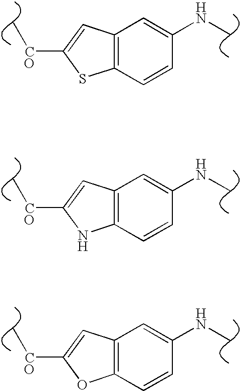

3. A compound represented by either of the following structures: 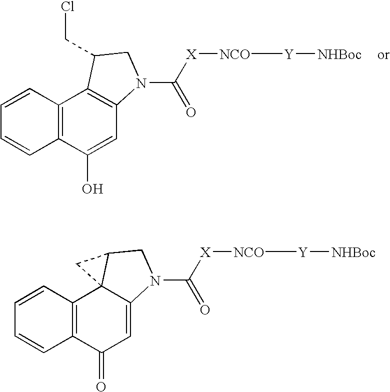
wherein —C(O)XN— is represented by the following diradical: 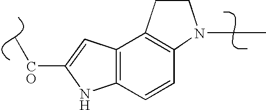
and —C(O)YNH— is selected from the group of diradicals consisting of:

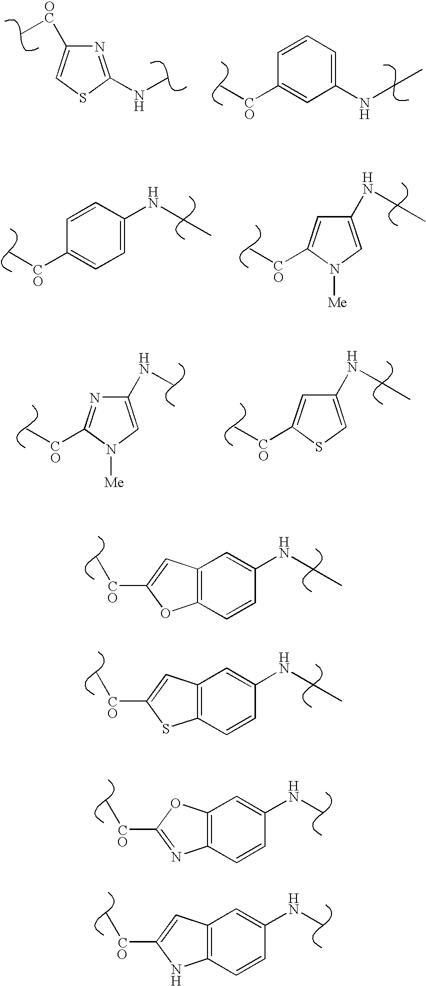

4. A compound represented by either of the following structures: 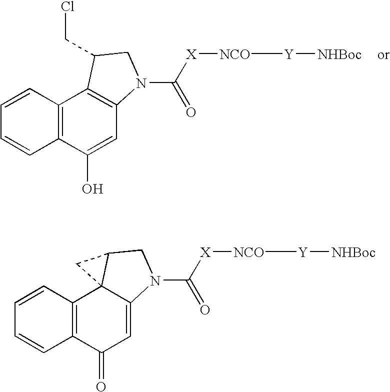
wherein —C(O)XNH— is selected from the group of diradicals consisting of: 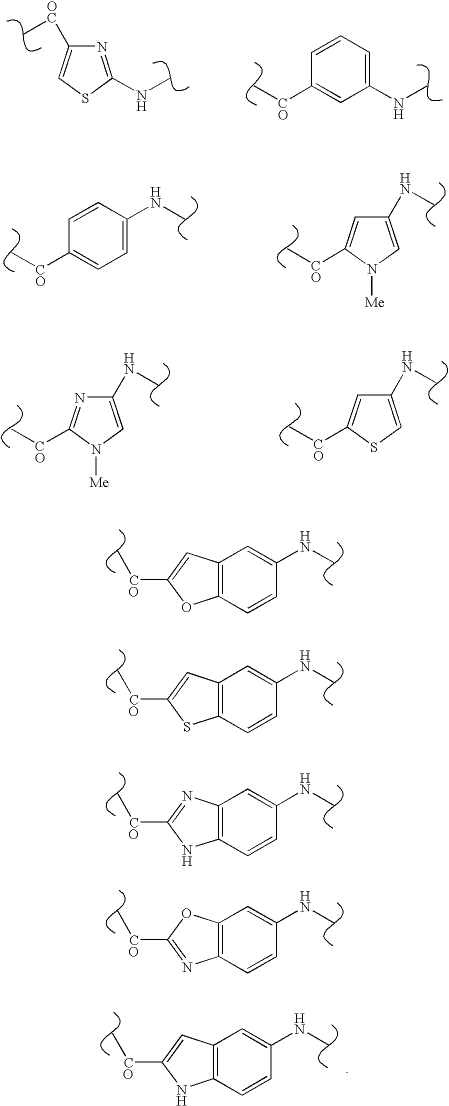
and —C(O)YN— is represented by the following diradical:

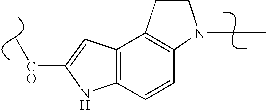

5. A process for killing a cancer cell comprising the step of contacting the cancer cell with a composition having a cytotoxic concentration of one or more of the compounds described in claims 1-4, the cytotoxic concentration being cytotoxic with respect to the cancer cell.
